(19) United States
(12) Patent Application Publication
(10) Pub. No.: US 2009/0175955 A1
(43) Pub. Date: Jul. 9, 2009

(54) HYDROPEROXYLAPATITE AND COMPOSITIONS THEREOF

(76) Inventors: Igor Anatolievich Pomytkin, Moscow (RU); Valentin Antonovich Vinogradov, Moscow (RU)

Correspondence Address: NOTARO & MICHALOS P.C., 100 DUTCH HILL ROAD, SUITE 110, ORANGEBURG, NY, 10962-2100, US

(21) Appl. No.: 12/296,566

(22) PCT Filed: May 17, 2006
(86) PCT No.: PCT/RU06/00245

§ 371 Date: Oct. 9, 2008

(51) Int. Cl. A61K 8/19

(52) U.S. Cl. 424/602

(57) ABSTRACT

This invention relates to a compound of general formula Ca₅(PO4)₃(OOH)ₓ(OH)₁₋ₓ wherein 0<x<1, and compositions thereof.

### TECHNICAL FIELD

The present invention relates to hydroperoxylapatite and compositions thereof.

### BACKGROUND OF THE INVENTION

Hydroxylapatite Ca₅(PO₄)₃OH is a major component of bone, dentine, and tooth enamel in mammals. Hydroxylapatite is frequently used in medicinal applications directed to treating bone, dentine, and tooth enamel (e.g. U.S. Pat. No. 6,254,855).

A variety of hydroxylapatite is known from the art, for example, fluor-hydroxylapatite Ca₅(PO₄)₃(OH, F), which hydroxylapatite is formed by partial replacement of hydroxyl-group by the other atom (e.g. fluorine). The complete replacement results in compounds with general formula Ca₅(PO₄)₃Y (e.g. Y is F, Cl, and etc.), which compounds are known from the art as apatites, for example, fluorapatite Ca₅(PO₄)₃F, chlorapatite Ca₅(PO₄)₃Cl, and etc. The replacement modifies hydroxylapatite properties and this modified material is frequently used in medicinal applications (e.g. U.S. Pat. No. 5,496,541).

Apatite compounds formed by partial or complete replacement of hydroxyl-group by hydroperoxyl-group of general formula Ca₅(PO₄)₃(OOH)ₓ(OH)₁₋ₓ (wherein 0<x≦1) are unknown from the art.

Oral care compositions or therapeutic compositions comprising the compound of general formula Ca₅(PO₄)₃(OOH)ₓ(OH)₁₋ₓ (wherein 0<x≦1) are unknown from the art.

It is an object of the present invention to provide a compound of general formula Ca₅(PO₄)₃(OOH)ₓ(OH)₁₋ₓ (wherein 0<x≦1) and compositions thereof.

### DISCLOSURE OF INVENTION

The present invention provides a compound of general formula (I)

Ca₅(PO₄)₃(OOH)ₓ(OH)₁₋ₓ (I)

wherein 0<x≦1. Preferably, x varies from 0.1 to 1. More preferably, x equals to 1 and the compound is Ca₅(PO₄)₃(OOH) or a pharmaceutically acceptable salt thereof.

The compound of the invention can be prepared by a process comprising a step of reacting calcium peroxide with phosphoric acid.

Preferably, phosphoric acid is added to the suspension of calcium peroxide in distilled water and the precipitate of the compound of the invention is filtered off and dried under vacuum. The compound of the invention Ca₅(PO₄)₃(OOH)ₓ(OH)₁₋ₓ can be prepared in the process further comprising a step of partial thermal decomposition of dry Ca₅(PO₄)₃(OOH) to provide the desired x.

In another preferred embodiment of the process of the invention, desired compound Ca₅(PO₄)₃(OOH)ₓ(OH)₁₋ₓ can be prepared by adding phosphoric acid to the suspension of mixture of calcium peroxide and calcium oxide taken in molar ratio providing the desired x. The resulting compound of formula Ca₅(PO₄)₃(OOH)ₓ(OH)₁₋ₓ is filtered off and dried.

Further, the present invention provides a composition comprising a compound of general formula Ca₅(PO₄)₃(OOH)ₓ(OH)₁₋ₓ wherein 0<x≦1 and an excipient. Such excipients include, but are not limited to, water, inorganic or organic solvents, inorganic or organic powders, and hydroxylapatite.

Further, the present invention provides an oral care composition comprising a compound of general formula Ca₅(PO₄)₃(OOH)ₓ(OH)₁₋ₓ wherein 0<x≦1 and a pharmaceutically acceptable excipient. Preferably, x varies from 0.1 to 1. More preferably, x equals to 1 and the compound is Ca₅(PO₄)₃(OOH) or a pharmaceutically acceptable salt thereof.

Further, the present invention provides a therapeutic composition comprising a compound of general formula Ca₅(PO₄)₃(OOH)ₓ(OH)₁₋ₓ wherein 0<x≦1 and a pharmaceutically acceptable excipient. Preferably, x varies from 0.1 to 1. More preferably, x equals to 1 and the compound is Ca₅(PO₄)₃(OOH) or a pharmaceutically acceptable salt thereof.

The compositions of the invention are prepared by known procedures and contain ingredients and excipients which are well known in the art. A generally recognized compendium of such methods and ingredients is Remington's Pharmaceutical Sciences by E. W. Martin (Mark Publ. Co., 15th Ed., 1975). In making the therapeutic compositions, the compound of formula Ca₅(PO₄)₃(OOH)ₓ(OH)₁₋ₓ, wherein 0<x≦1, will usually be mixed with appropriate excipient and formulated in a variety of unit dosage form. Such forms include, but are not limited to, suspensions, gels, water dressings, and pastes including tooth pastes.

The compound of the invention is generally used in the composition at levels from about 0.05 to 90.0%, preferably from about 3 to 50% by total weight of the composition. The preferred excipient of the invention is water which is generally used in the composition at levels from about 10 to 99.5%, preferably from about 50 to 97% by total weight of the composition.

Preferably, the size of particles of the compound of the invention is from 0.01 to 100 μm.

The composition of the invention can comprise non optional ingredients. Such ingredients include, but are not limited to, surfactants, stabilizers, colorants, and flavors.

Non-exclusive examples of stabilizers include, but are not limited to, sodium fluoride, potassium fluoride, and calcium fluoride.

The composition of the invention is provided in the pH range from about 5.5 to 11. The more preferred pH range is about 7 to 9. Preferably, buffer of the invention include, but are not limited to, glycine, citrate, succinate, fumarate, malate, bicarbonate, or phosphate buffer.

The compositions of the invention can be used in methods for treating tooth enamel and dentine, which comprise a step of administering to a mammal in need thereof said compositions to achieve a desired therapeutic effect. Preferably, mammal is a human.

In practicing the invention, the effective amount of the composition of the invention is administered into oral cavity to achieve a contact between the compound of the invention and a surface of tooth enamel and/or dentine and retaining said composition in the oral cavity for at least 5 seconds. Preferably, the retaining time is 2 min, more preferably is 15 min. The effective amount of the composition of the invention is from 0.1 to 10 g, preferably from 0.5 to 1 g. Because of oxidant properties and similarity of the compound of the invention with the natural material of tooth enamel and dentine, a variety of benefit therapeutic effects are achieved with the compositions of the invention. Such effects include, but are not limited to, bleaching the tooth enamel, repairing the tooth enamel and/or dentine, treating carious cavity, antimicrobial treating of oral cavity, treating gingivitis, treating periodontal disease, and freshening breath.

Further, the compositions of the invention can be used in methods for treating bone, which comprise a step of administering to a mammal in need thereof the therapeutic composition comprising a compound of formula Ca₅(PO₄)₃(OOH)ₓ(OH)₁₋ₓ.

In practicing the invention, the effective amount of the therapeutic composition of the invention is administered to a bone to achieve a contact between the compound of the invention and the bone. The effective amount of the composition of the invention is from 0.1 to 100 g, preferably from 1 to 10 g. Because of oxidant properties and similarity of the compound of the invention with the natural material of bone, a variety of benefit therapeutic effects are achieved with the method of the invention. Such effects include, but are not limited to, accelerating bone repairing after fractures and antimicrobial treating of bone fracture field.

Further, compositions of the invention can be used in methods for wound healing, which comprise a step of administering to a mammal in need thereof the therapeutic composition comprising a compound of formula Ca₅(PO₄)₃(OOH)ₓ(OH)₁₋ₓ.

In practicing the invention, the effective amount of the composition of the invention is administered into wound to achieve a contact between the compound of the invention and the wound surface and retaining said composition in the wound for at least 10 min. Preferably, the retaining time is 1 hour, more preferably is 24 hours. The effective amount of the composition of the invention is from 0.1 to 10 g, preferably from 0.5 to 1 g. Because of oxidant properties and similarity of the compound of the invention with the natural material of bone, a variety of benefit therapeutic effects are achieved with the method of the invention. Such effects include, but are not limited to, accelerating wound healing, accelerating bone repairing, and antimicrobial treating of wound.

The following examples are presented to demonstrate the invention. The examples are illustrative only and are not intended to limit the scope of the invention in any way.

### EXAMPLE 1

This example demonstrates the process for preparing the compound of the invention.

A solution of 0.7 ml of 85% phosphoric acid was added slowly by drops to the suspension of 0.72 g calcium peroxide in 25 ml distilled water at ambient temperature to give the compound of formula Ca₅(PO₄)₃(OOH) on standing. The precipitate is filtered off and dried under ambient temperature in vacuum. Analysis: Ca 38.6%, P 17.9%, —OOH 6.4%.

Then, the compound of formula Ca₅(PO₄)₃(OOH)_{0.1}(OH)_{0.9} is prepared by controlled decomposition of the starting compound of formula Ca₅(PO₄)₃(OOH) in vacuum at rising the temperature with rate of 1 degree/min and continuous monitoring of the sample mass. The loss of 2.8% of starting mass means that the desired compound has prepared. Analysis: —OOH 0.65%.

### EXAMPLE 2

This example demonstrates the process for preparing the compound of the invention.

A solution of 0.7 ml of 85% phosphoric acid was added slowly by drops to the suspension of 0.28 g calcium oxide and 0.36 g calcium peroxide in 25 ml distilled water at ambient temperature to give the compound of formula Ca₅(PO₄)₃(OOH)_{0.5}(OH)_{0.5} on standing. The precipitate is filtered off and dried under ambient temperature in vacuum. Analysis: Ca 39.3%, P 18.2%, —OOH 3.2%

### EXAMPLE 3

This example demonstrates the oral care composition of the invention.

| | % by weight |
|---|---|
| Ca₅(PO₄)₃(OOH)_{0.1}(OH)_{0.9} | 7 |
| Sodium fluoride | 0.6 |
| Distilled water | 92.4 |

The method for preparing the composition described in Example 3 was as follows: the compound of the formula Ca₅(PO₄)₃(OOH)_{0.1}(OH)_{0.9} and sodium fluoride were mixed with distilled water to form suspension.

The composition is administered into oral cavity to achieve a contact between the compound of the invention and a surface of tooth enamel and/or dentine and retaining said composition in the oral cavity for 10 min to provide the effect of tooth bleaching

### EXAMPLE 4

This example demonstrates the therapeutic composition of the invention.

| | % by weight |
|---|---|
| Ca₅(PO₄)₃(OOH)_{0.1}(OH)_{0.9} | 7 |
| Distilled water | 93 |

The method for preparing the composition described in Example 4 was as follows: the compound of the formula Ca₅(PO₄)₃(OOH)_{0.1}(OH)_{0.9} was mixed with distilled water to form suspension.

The composition is administered to a bone fracture area to achieve a contact between the compound of the formula Ca₅(PO₄)₃(OOH)_{0.1}(OH)_{0.9} and the bone to provide the effect of acceleration of the bone repairing.

We claim:

1. A compound of general formula (I)
Ca₅(PO₄)₃(OOH)ₓ(OH)₁₋ₓ (I)
wherein 0<x≦1.

2. A composition comprising the compound of claim 1 and an excipient.

3. An oral care composition comprising the compound of claim 1 and a pharmaceutically acceptable excipient.

4. A therapeutic composition comprising the compound of claim 1 and a pharmaceutically acceptable excipient.
